(19) United States
(12) Patent Application Publication
(10) Pub. No.: US 2022/0134061 A1
(43) Pub. Date: May 5, 2022

(54) Systems for Delivery of Therapies by Trans-maxillary Access

(71) Applicant: Akshal Patel, Seattle, WA (US)

(72) Inventor: Akshal Patel, Seattle, WA (US)

(21) Appl. No.: 17/512,982

(22) Filed: Oct. 28, 2021

Related U.S. Application Data
(60) Provisional application No. 63/108,327, filed on Oct. 31, 2020.

(51) Int. Cl. A61M 25/10; A61M 25/09; A61K 31/727; A61K 31/167; A61K 31/4545

(52) U.S. Cl. CPC A61M 25/10; A61M 25/09; A61K 31/727; A61M 2025/091; A61K 31/4545; A61M 2025/105; A61K 31/167

(57) ABSTRACT

Trans-maxillary access systems, devices and appliances governed by AI sensing and data arrays are shown, along with analogous ingress through occipital artery and related vasculature. Likewise, occipital artery access follows the same paradigm, procedures and protocols for data. Contractomeres overcome drawbacks when used with the instant systems, processes, and methodologies.

### RELATED APPLICATIONS

This application claims the full Paris Convention priority rights, benefits and privileges to U.S. provisional patent application No. 63/108,327, filed on Halloween Day; Oct. 31, 2020. This substitute specification does not add any new matter pursuant to 37 CFR 1.125(b).

### BACKGROUND OF THE INVENTIONS

### Field of the Technology

The present invention introduces a new paradigm for treating chronic subdural hematomas requiring access to the middle meningeal artery (MMA), specifically, a more direct method for entry into the head and neck is for the first time being set forth by this application for U.S. Letters Patent.

Likewise, the present invention relates to systems and method for inserting a catheter into the human body, although the superficial temporal artery (STA) has yet to be used as a retrograde access side to the middle meningeal artery (MMA) and associated vasculature.

The present disclosures relate to neurovascular remediation, in particular the instant application is believed to introduce the conceptual paradigm shift defined as “trans-maxillary access” as depicted for example in FIG. 1, disclosed and claimed below.

The present invention relates to access to and delivery of therapies to the brain, and generally speaking this includes many instances on an instantaneous basis and with high risks. Further included are lower risk and ostensively out-patient procedures, under the new paradigm presented.

Offered for consideration, including all required information for the below-listed patents, is a novel approach to endovascular, neurovascular, and cardiovascular surgical access which overcomes pitfalls of cervico-cephalic platforms. Instead, the disclosure herein teaches trans-maxillary access to the target vasculature.

The following U.S. Letters Patent are expressly incorporated herein by reference, as if fully set forth in their entirety: U.S. Pat. Nos. 10,398,877; 9,808,359; 9,566,072; 9,220,522; 9,387,098; 9,566,071; 8,945,143; 8,926,680; 8,070,791; 8,088,140; 8,197,493; 8,574,262; 8,585,713; 6,824,558; 6,306,141; 6,302,906; 6,117,167; and, 6,051,020. Applicant submits an Information Disclosure Statement reciting these references, herewith.

### Objects and Summary of the Invention

Briefly stated, trans-maxillary access systems, devices and appliances governed by AI sensing and data arrays are shown. Data groupings and blockchain-like systems for sharing and compliant transmission of medical imaging data are likewise contemplated.

### EXAMPLE FIRST—CALCIUM CHANNEL BLOCKER CREAM APPLIED

Inserting a catheter system into the superficial temporal artery (STA), which sits just under the surface of the scalp, as a retrograde access site to the middle meningeal artery (MMA) is a direct route not yet taken to date. It is respectfully proposed that neither equipment nor devices exist for the newly proposed portal of entry into the cranial arterial network.

The present inventor has now performed a first-in-man procedure, whereby the common technique for introducing a catheter into the vasculature, via direct STA and MMA access, was achieved and using ultrasound to identify precise location, a hematoma was treated.

The first in human experience which occurred on or about June 24 and 25, 2021, is embodied in the claimed process for delivery of therapies by trans-maxillary access and all steps set forth. Artisans understand ultrasound usage in conjunction with same, and any related imaging needed.

Variations on the Seldinger technique, invented by a Swedish radiologist in 1953, after whom it is named, have yet to apply it to the STA. Making an incision in the arterial wall after applying calcium channel blockers, enables retrograde access to the cranial arterial network.

According to embodiments, there is provided trans-maxillary access systems, therapies, closure devices and related specialized devices and appliances. Infusion of thrombolytic and non-thrombolytic agents may be achieved with balloon catheters and likewise through a pump/reservoir system docked on the scalp.

According to embodiments, there are shown methods of making and using catheter systems, wires, and AI data compiling sensors and data sets. Likewise, systems are taught for retrograde access to the cranial vasculature along with state of the art sensing systems, and visualization means applied to those systems.

According to the teachings of the present invention, specific sets of catheters having unique caliber and length, with appropriate flexibility to work within the confines of the spaces inside the vasculature, are taught.

According to embodiments, automated and AI-driven specialized and general purpose computing means integrate innovative procedures with clinically significant and successful outcomes for patients, with risks mitigated.

### BRIEF DESCRIPTION OF THE DRAWINGS

Various preferred embodiments are described herein with references to the drawings in which merely illustrative views are offered for consideration, whereby:

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

Various objects, features, aspects and advantages of these inventive subject matters will become apparent from the following detailed descriptions of operative and enabled embodiments, along with the schematic cartooned and line drawing figures, in which, (as reference designators show) the anatomy and devices and method of the present invention—to the extent possible—like numerals represent common vessels and components.

FIG. 1 shows a schematic cartoon of a flow chart outlining select novel steps and aspects of the teachings of the present invention.

FIG. 2 shows the STA, and its location on the human scalp and the MMA, plus gateways to the salient cranial vasculature, including the occipital artery (OA) pathways;

FIG. 3 illustrates a detailed view of a lesion along the frontal branch of the STA, along with a view of the vessel dilated by calcium channel blocker, to make it easier to catheterize, for treatment of chronic subdural hematoma;

FIG. 4 shows the vast potential, including for main, sets of directional travel which are first disclosed herein as novel procedures flowing retrograde from the STA, as process of FIG. 1 and devices of all subsequent figures show, claimed below as the Objects of the present invention;

FIG. 5 likewise is a cartoon to represent a balloon catheter, along with supporting sheaths, introducers and an infusion line to elute pharmaceuticals, noting over-the-wire from incorporated patents, into the ICA, to the ECA, according to the present invention;

FIG. 6 demonstrates internal maxillary artery access to the balance of the front of the head, and MMA's access to more cranial vasculature;

FIG. 7 shows a schematized port for infusing long-term medications, as well as a transponder for sending/receiving signals, docked on the scalp (See FIG. 2); and

FIG. 8 shows that specific catheters are designed for the processes and functions of the present inventions. Meanwhile, FIG. 9 shows the expansible and contractile nodes of the catheter which will be referred to as contractomeres.

### DETAILED DESCRIPTIONS

Treatment of chronic subdural hematomas (CSH) requires an enhanced therapy set owing to the challenges of trans-femoral and evolving radial access.

It is respectfully proposed that the present inventor introduces for the first time access devices to the external and internal carotid artery circulation, all branches, as well as specialized catheters adapted to these intracranial pathology, addressing at least the range described and claimed below.

It is further respectfully submitted that U.S. Letters Patent are appropriate for introducing Trans-Maxillary, as opposed to traditional (prior art cervico-cephalic) approaches, as shall become patent from the description below.

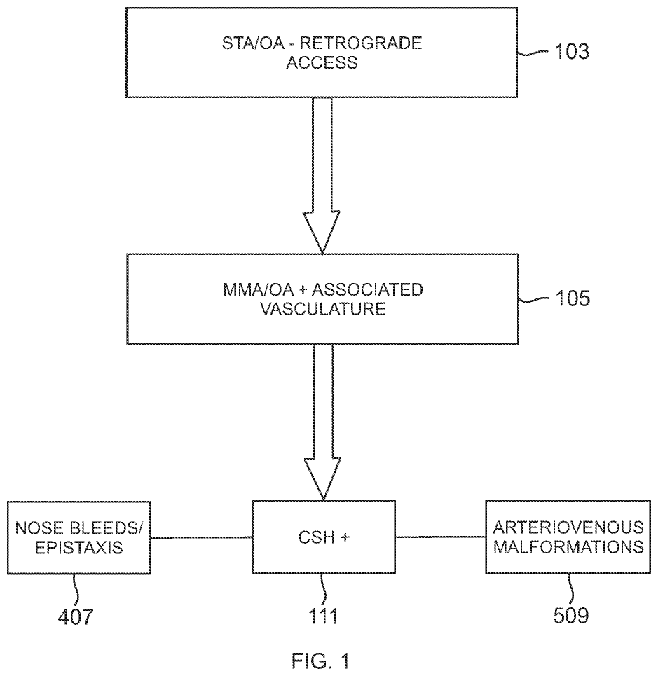

Referring to now, to FIG. 1 those of skill in the art understand and can have located the superficial temporal artery (STA) 103 which sits just under the surface of the skin of the scalp. The new paradigm involves use of this portal of entry used as retrograde access to the middle meningeal artery 105 (MMA), and associated vasculature.

The new approach and each new tool provide a more direct method for entry into the head and neck region, for all manner of stenting means, and all tools and devices needed.

Referring now to FIG. 1, cranial access through the superficial temporal artery (STA) 103 is undertaken following the application of cream comprising calcium channel blocker 111, which makes the superficial temporal artery (STA) dilated and easier to catheterize, as known to those skilled in the art; just as, see, for example, U.S. Pat. No. 10,398,877. Access for endovascular sites was known, not including trans-maxillary access, until the advent of this filing. It is respectfully proposed that each said teaching likewise works with the occipital artery (OA) extension of this platform/paradigm approach.

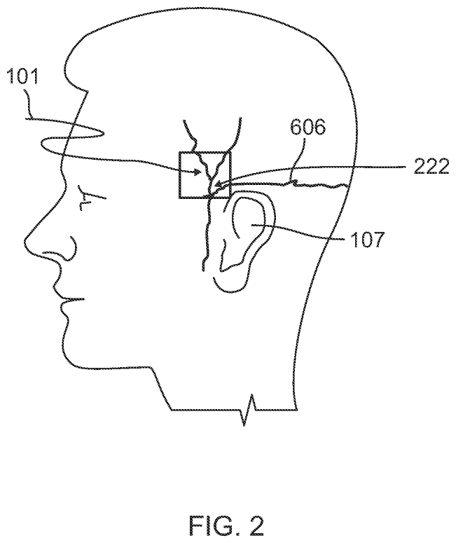

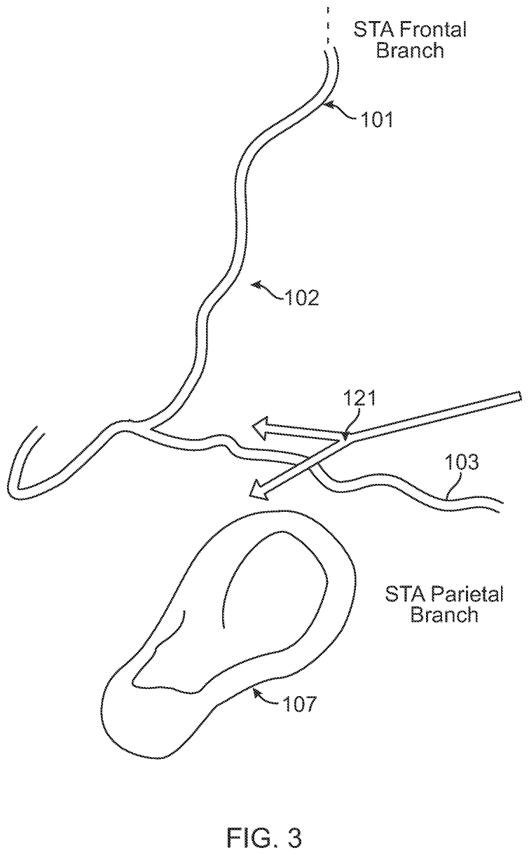

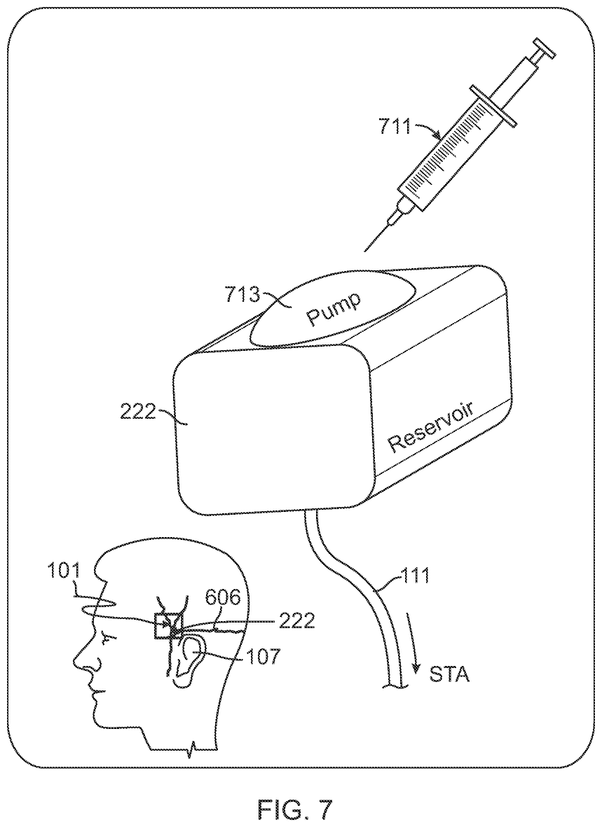

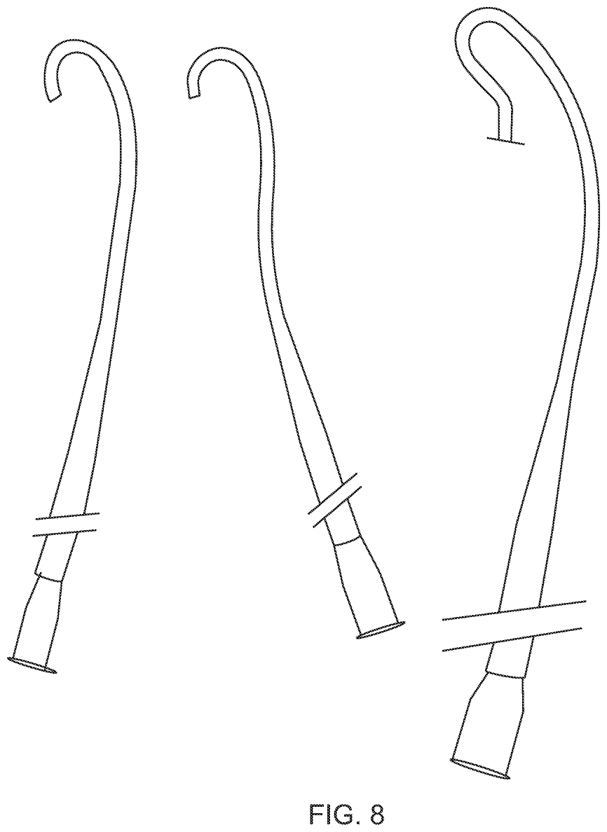

Referring now also to FIGS. 2-4, the location of the superficial temporal artery (STA) 101 is known, and also is identified by ultrasound as needed to identify its precise location in the scalp, emerging proximate to the mammalian ear 107, or behind the ear as occipital artery (OA) 606, as shown in Figure, and the first being shown in FIG. 3 in detailed view, with lesion 102. FIG. 2 likewise shows 222 as a location of port for long-term medication (as discussed in FIG. 7) such as calcium channel blockers.

Referring now to FIG. 3, cream 111 is applied before surgery which makes the superficial temporal artery (STA) 101 dilated and easy to catheterize. This also applies, as discussed, in lockstep with the occipital artery (OA). Again, Artisans have known using endovascular teachings from U.S. Pat. Nos. 6,051,200 through 9,808,359, and 9,566,071, to address the need to dilate the vessel for catheterization.

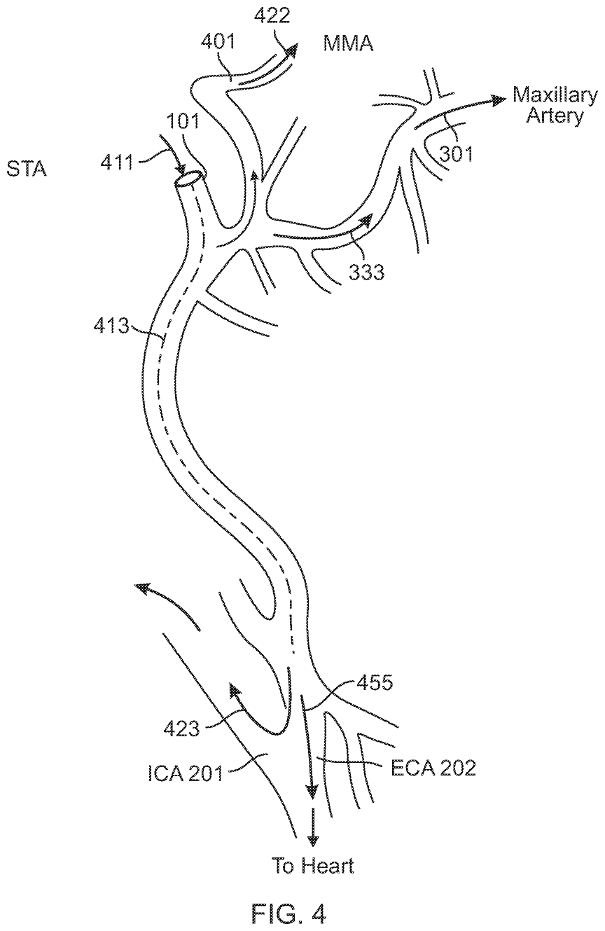

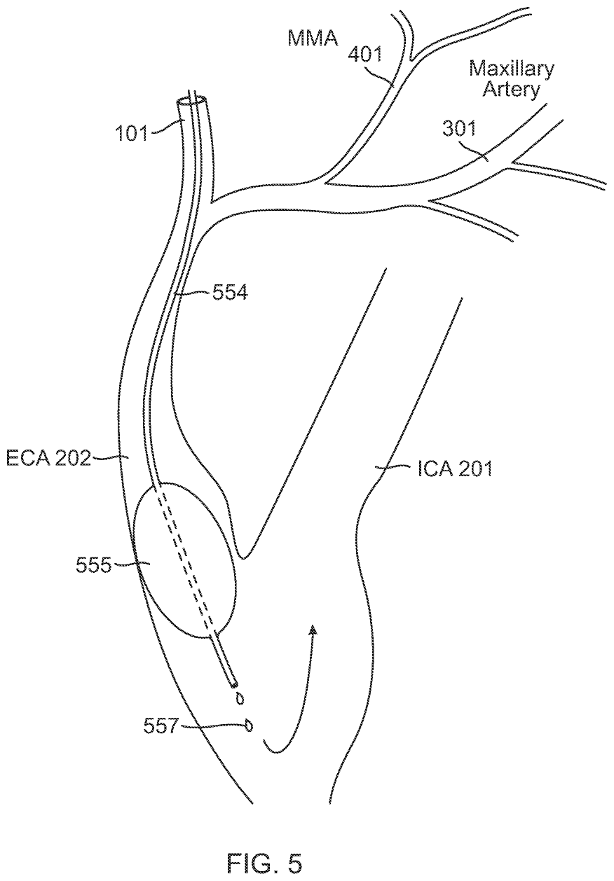

FIG. 3 likewise shows that lesion 102 can be pinpointed in the scalp by ultrasound, and that forked medial device appliance, or functionally-similar apparatus 121 secures or pins either principal vessel 101 the superficial temporal artery (STA), or branch 103, the superficial temporal artery (STA) parietal branch so that the superficial temporal artery (STA) is stabilized, not moving, to allow wire/microwire penetration, as known. Novel again is the retrograde approach here. See also FIG. 4, wherein arrow 411 shows the direction of travel for the systems of the present invention retrograde (arrow to the dotted line 413) in the direction of heart 455 and coronary vessels. Internal carotid artery (ICA) 422, and arrow 423 showing this direction of travel, reveals that ICA 422, and ECA 401 comprises the untapped highway of the circulation (See FIG. 5). FIG. 4 shows the middle meningeal artery (MMA) 401, and direction of travel 422, along with maxillary artery 301 and direction of travel 333 to the maxillary system further comprising nose, face, and eyes (not shown) 333.

It is posited—although data-based evidence shall determine outcomes—that as offered for consideration, a retrograde surgical therapy delivery set comprising both systems and processes enable the multiple directions summarized well by FIGS. 4, 5 and 6, namely, internal carotid artery (ICA) 201, external carotid artery (ECA) 202, perhaps offers the largest number of untapped procedural iterations.

FIG. 5 shows an example of balloon catheter 555, which is positioned to sit in external carotid artery (ECA) 202, introduced from retrograde access from superficial temporal artery (STA) 101. Those skilled in the art understand medications can then be eluted into the circulation directed and positioned to travel into the internal carotid artery (ICA) 201. Likewise, in addition to their primary role supplying the viscera and the head and neck and the dura, branches of the external carotid artery (ECA) 202 are important sources of collateral blood flow in cases of external carotid artery (ECA), internal carotid artery (ICA), and vertebral artery stenosis or occlusion.

Said collateral pathways take various forms (not shown) from internal carotid artery (ICA) to external carotid artery (ECA), from external carotid artery (ECA) to internal carotid artery (ICA), and external carotid artery (ECA) to vertebral artery. Flow may pas in either direction depending on the pattern of vascular stenosis or occlusion.

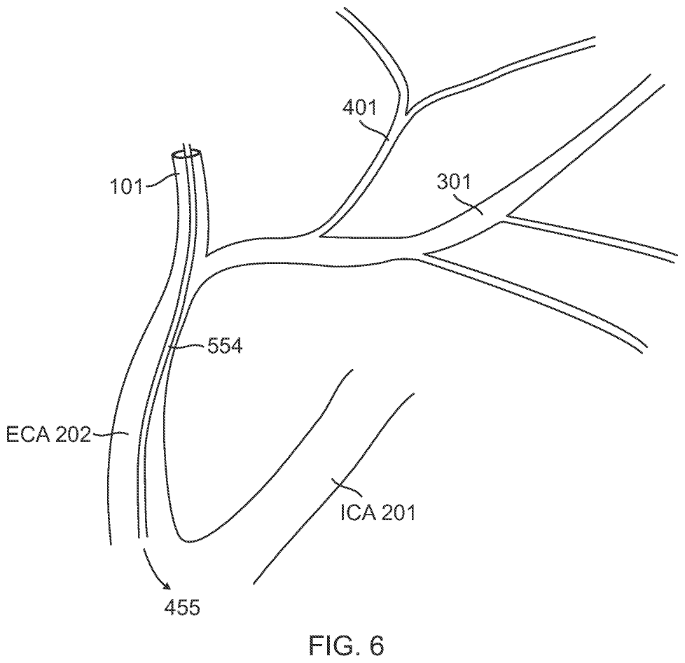

FIG. 6 likewise shows how the superficial temporal artery (STA) 101 supplies the scalp, branches of the internal maxillary artery 301, and supply the cranial dural matter and middle meningeal artery (MMA), extracranial musculature (deep temporal arteries) sino-nasal region and maxillary alveolus, and palate and portions of the face. The same as occipital artery (OA) services the entire rear side of the cranial coruscation, working lockstep with the STA access, and all descripts from one are herein applied definitionally to the other, in terms of function, structure, and results, and substantially similar equivalents.

FIG. 7 shows reservoir/port 222 with neck access 711, pump 713, and catheter linked to the superficial temporal artery (STA) 101.

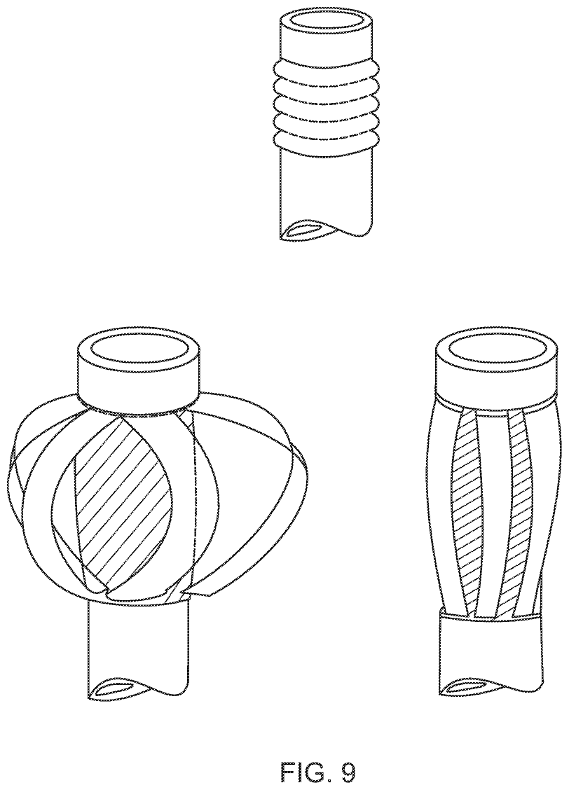

FIG. 8 likewise offers three views of distinctly curved catheter segments with specialized durometers, linings and coatings, which work with known uses (not shown). However, those of skill in the art can appreciate that new approaches to vessels require specialized wire configurations and catheter tools to accomplish. Shown in FIG. 8 are three base curves, which are modified and used with balloons (see FIG. 5) stent-retrievers/flow diverters (see above) and related tools.

FIG. 9 shows the expansible and contractile nodes of the catheter which will be referred to as contractomeres. The contractomeres are the aspiration mechanism which will provide a sinusoidal positive and negative pressure to allow the catheter to ingest highly viscous slurry materials, such as clot.

Contractomeres are designed with three elements; a coiled spring (not shown) sits in the wall and can be made up in a single or double helical configuration. It is made of stainless steel and will store energy during negative vacuum. The second is the pleated jacket, which can match the helical or screw pitch of the spring. It is constructed of a biocompatible polymer such as polyethylene and will be reinforced with Nitinol braids to provide strain relief and reduce kinkability. The inner surface is coated with silicone composite material to make it lubricious. The jacket is to deform and collapse under axial load much like an accordion. The third and outermost component is the proleg, which is an extension of the non-contractomere or fixed component of the catheter which envelopes the jacket and spring. Proleg is a leaf-spring that bends outward and can temporarily anchor the catheter to the inner lumen of the vessel wall.

During periods of “rest” or non-aspiration conditions, proleg is flush with the main catheter and appears as a longitudinally vertebrated portion of the main catheter. During collapse of the contractomere, under axial load, proleg components bend outward, fattening the diameter of the catheter.

The contractomere's jacket and spring are made separately and later bonded to the catheter. The proleg is made by barrel staving the catheter along its axis. Each contractomere is calibrated to displace at a specific force and thus a unique kinomatic profile, though there is overlap of activation of each contractomere during an aspiration cycle.

In the setting of an occluded artery, the catheter will sit in close proximity to the obstruction. With this arrangement the artery and catheter together act as a fixed hydrostatic system, with a relatively unchanged fluid volume. With cyclical aspiration, the catheter will inch toward the occlusion.

The aspiration mechanism is a device that is attached directly or indirectly to the end of the catheter. It will provide sinusoidal vacuum forces that will cause repetitive loading and unloading of forces on the contractomere. The frequency of the aspiration wave can vary to augment or retard the inching of the catheter. There can be plateaus of negative pressure or positive pressure at the discretion of the operator. A low and high frequency variance changes the activity of the catheter. Low frequencies are more likely to cause forward progress, high frequencies are more likely to cause break-up of occlusive materials at the end of the catheter.

The catheter will be used as a platform for other devices deployed or in preparation for deployment in the artery. In the setting of occlusion: stents, retrievers, stent-retrieving combinations, balloons and separators can all be used in concert with the invention. These devices, commonly used in stroke and pulmonary embolism, will act as distal anchors for the “crawling” catheter. During catheter motion, it will invest and ingest the occlusive material, and ride over the anchoring devices to envelope them as well.

The advantages of this catheter are related to its use in the vascular system. Where the peristalsis of the catheter generates an atraumatic anterograde advancement. Inside the muscular artery this retains a physiologic profile in the setting of aspiration or retrieval of occlusive materials. Traditional catheters act as fixed tubular skeletons and can create force vectors that damage vessel lumens. The invention is modular and takes into account the elastic property of the artery wall.

GLOSSARY. In directionality embodiments of the system, medical device, catheter, sheath, combination of catheter and sheath, guide wire, combination of guide wire and catheter, and methods of the present disclosure, the term “proximal” refers to regions of medical device that is closest to end of catheter that has a hub, while the term “distal” refers to region that is farthest away from hub.

Flipped-tip segment residing at the distal end of catheter. Any catheter embodiment of the present disclosure can include, at the distal end, an additional segment (segment that is even more distal) taking the form of a flipped tip. Flipped-tip consists of a short straight segment that is coupled to a short curved segment. Curved segment assumes a curve that defines a 45 degree angle, and where the curve that assumes a 45 degree angle that has a central point that is near to (rather than, relatively far away from) a long curved segment that is immediately proximal to the flipped-tip. In other words, flipped-tip is distal and long curved segment is proximal, where “distal” means relative far from the hub, and “proximal” means relative close to the hub.

In catheter embodiments that do not include any hub, the term “proximal” can refer to the end of the catheter that is farther from patient's heart immediately after the catheter is inserted into patient's vasculature, and the term “distal” can refer to the end of the catheter that is closer to patient's heart immediately after the catheter is inserted into patient's vasculature.

Endovascular techniques are predominantly driven by specialists based on organ system and have been refined by them as techniques evolve. For instance, initial arterial endovascular approaches were done by puncturing the neck artery. Due to the potential for injury to that vessel, endovascular approaches subsequently developed into puncturing the groin artery (transfemoral approach). For doctors addressing the arteries of the heart, over the past decade the majority have been accessing across the wrist artery (transradial approach), as the approach is more direct, substitutes the higher risk of groin access complications for the lower risk of wrist issues, and leads to increased patient satisfaction and shorter, improved post-procedural recovery.

Neuro-endovascular procedures, those dealing with the brain's vasculature, still predominantly utilize the transfemoral approach. The majority of neuro-endovascular procedures are performed in the younger and middle-aged population, lending to the ease of the transfemoral approach to access all the vessels of the head and neck. Due to the advancing patient age from the increasing number of acute stroke therapies, the vasculature conformation is increasingly becoming more difficult via the transfemoral approach. Concomitantly, the need for larger catheters and the increasing use of stronger blood thinning agents (agents that impair blood clotting) required for some procedures, both increase the life-threatening nature of groin access complications.

Advantages of Systems, Devices, Catheters, and Methods of the Present Disclosure: Without implying any limitation, the present disclosure provides a number of advantages. Advantages of each of the inventive curves and shapes of the catheter are that they allow for optimal positioning to make the turns into the right common carotid artery; furthermore, to provide a stable tip curve to first allow a wire to be advanced through the catheter up as high as needed, then taking advantage of the design optimization in its design-in-totality with the wire stiffness and diameter once the wire is fully advanced, can then be advanced over the wire until the catheter's tip is in a desired location in the neck artery.

Another advantage is reduced adverse events, including reduced neurological adverse events. These include reduced neurological adverse events due to scraping of the top of aortic arch where calcium and cholesterol tend to deposit, where the scraping occurs when advancing a catheter trans-femorally up into vessels that pass through vessels residing in the neck and in the brain.

ADVANTAGE OF THE FLIPPED-TIP: The distal region of the catheter, which may be called a “shepherd's hook” or a “candy cane hook,” possesses a 45-degree bend (the flipped-tip). This 45 degree bend provides the advantage in some circumstances of providing extra engagement by positioning the distal region of the catheter on the wall of the artery that is most likely to have an origin of the target artery.

Methods for Manufacturing Catheter of the Present Disclosure: The present invention comprise a method for manufacturing catheter. The main components of the catheter include, a tube made of a polymer, a coil winding, a mix of stainless steel, and a nitinol hydrophilic coating.

For manufacturing, it all starts with the mandrel. The mandrel is like a scaffold. Mandel has a PTFE liner coating on it. The mandrel is cut to size and placed in a machine with spools of stainless steel and nitinol. The machine applies the stainless steel and nitinol coil winds on top of the PTFE liner on the mandrel. The pattern is a single wire of stainless steel followed by three wires of nitinol with varying thicknesses (this is one embodiment only). The catheter is typically without coil winding at the distal 1-1.5 cm. This nitinol-stainless steel pattern repeats on the proximal shaft. At the distal end of the catheter, the construction is completely nitinol. There may also be laser welding joining nitinol and stainless steel. Once the metal is applied correctly, the polymers are applied. Polymer sections of varying stiffness according to the desired flexibility of the catheter section are placed over the metal. These polymers are then bonded to the catheter section underneath it with heat treating (usually it is suspended top to bottom to allow the heat set to bind the coil to the polymer). A hydrophilic coating is applied to the distal section, via dipping with a mandrel inside, usually on the distal aspect, but typically not at the segment that interacts with the arch, approximately 30 mm section, halfway along the catheter; for our design, we may need to dip the shaft to coat it with the antivasospasm agent (see below). The final catheter has three layers from hub to tip. From inside to outside, there is a PTFE liner section, a metal coil wind section, and a polymer section. Typically, a hub can be affixed, and the hub is optional.

In exclusionary embodiments, system, medical device, catheter, and methods of the present disclosure can exclude any medical device that comprises, stainless steel, nitinol, tantalum, ceramic, nickel, titanium, aluminum, polymeric materials, stainless steel, titanium, niobium, or gold.

Coating methods; chemical reagents for coating. Coating can be via spray-coating, dip-coating, brushing, or vacuum-deposition.

Unique Geometry of the Catheter. A unique aspect of the catheter is that there is a variable geometry to the catheter to allow for the two different optimal conformations. The sheath and catheter are optimized and minimized difference between the inner diameter and the outer diameter. Manufacture as mentioned above may allow this to occur without sacrificing catheter strength and trackability.

Having a distal wire tip that is optimized/minimized in its outer diameter for insertion farther into the artery as it enters into the head/skull, while having a much larger transition to a larger diameter shaft, to present a stiff approach as it makes a turn in the arch or above it (as in a bovine left carotid artery), over which a catheter and/or sheath can be advanced over it without concern of “herniating” (or translating the pushing forces) the catheter out further into the arch, which is a hazard.

Coating of Anti-Vasospasm Drug on the Catheter's Shaft. The anti-vasospasm aspect of the catheter is in one embodiment a coating that prevents vasospasm, and lies over or in conjunction with the hydrophilic coating along any if not most of the shaft, not necessarily the tip. The drug can also be in a hydrogel. The drug can have the molecule coating the catheter. The drug can also be injected from the sheath sidearm and pass through a small channel(s) or rivulet(s) or laser cut holes (that allow injected drug from the sheath sidearm to to pass through the laser cut holes but does not allow blood to enter) running along the shaft of the catheter or sheath. An alternate embodiment of the anti-vasospasm drug takes the form of a time-released formulation.

Coating can be made from, for example, polyesters, polyimides, nylons (polyamides), polytetrafluoroethylene (PTFE), ethylene chlorotrifluoroethylene (ECTFE), perfluoroalkoxy (PFA), polyethylenes, polypropylenes, polyether block amide (PEBA) such as a Pebax®, polyurethanes, and so on. In exclusionary embodiments, system, medical device, catheter, of the present disclosure can exclude any system, medical device, or catheter that comprises one or more of the above chemicals.

Coating and Impregating Medical Device. The present disclosure provides a formulation for applying to a surface of a medical device, for example, by soaking, where the formulation comprises a dissolved plastic polymer. The dissolved plastic polymer can be more or more of or any combination of, polyurethane, polyethylene, polyethlyene teraphthalate, ethylene vinyl acetate, silicone, tetrafluoroethylene, polypropylene, polyethylene oxide, polyacrylate, and so on. What is encompassed are coatings, coating solutions, and medical devices that are coated with coating solutions, using Carbothane® family of polycarbonate-based aliphatic and aromatic polyurethanes, Estane®, which is a thermoplastic polyurethane, Pellethane®, which is a family of medical-grade polyurethane elastomers and exceptionally smooth surfaces, Tecoflex®, which is a family of aliphatic polyether polyurethanes, where low durometer versions are particularly suitable for long-term implant applications, Tecothane®, an aromatic polyurethane, Texin®, an aromatic polyether-based polyurthane which allows for very thin gauges (Microspec Corp., Peterborough, N.H.; Lubrizol, Inc., Wickliffe, Ohio; Entec Polymers, Orlando, Fla.). See, U.S. Pat. No. 6,565,591 of Brady, U.S. Pat. No. 7,029,467 of Currier, and U.S. Pat. No. 7,892,469 of Lim, which are incorporated by reference in their entirety. In embodiments, the present disclosure provides the recited polymers for use in coating solutions, or for use in manufacturing the medical device that is to be coated. A reagent, such as an anti-vasospasm agent, can be bulk distributed in the medical device, for example, by adding to a melted polymer or by soaking until even distribution has occurred.

Alternatively, medical device can be impregnated or coated with the agent. In embodiments, the disclosure encompasses methods for bulk distribution, gradient distribution, and limited surface distribution. Methods for manufacturing medical devices where an agent is bulk distributed, gradient distributed, or limited surface distributed, are available (see, e.g., U.S. Pat. No. 4,925,668 issued to Khan, et al, U.S. Pat. No. 5,165,952 issued to Solomon and Byron, and U.S. Pat. No. 5,707,366 issued to Solomon and Byron, all of which are incorporated herein by reference).

Measuring flexibility of entire catheter or of a segment of catheter. Flexural Modulus determines how much a sample will bend when a given load is applied, as compared to Tensile Modulus which determined how much a sample will stretch when a given load is applied and Compressive Modulus which determines how much a sample will compress when a given load is applied. Procedures for testing flexural modulus include, ASTM D790 and ISO178 (see. Beetle Plastics (Apr. 11, 2013) Testing and measuring flexural modulus. Ardmore, Okla.). Flexural Modulus by ASTM D790 or by ISO78 can be measured using a ZwickRoell testing machine (see, ZwickRoel, Kennesaw, Ga.). In embodiments, entire catheter, or a given segment of catheter of the present disclosure can have a Flexural Modulus of about 2 kpsi, about 4 kpsi, about 6 kpsi, about 8 kpsi, about 10 kpsi, about 12 kpsi, about 14 kpsi, about 16 kpsi, about 18 kpsi, about 20 kpsi, about 22 kpsi, about 24 kpsi, about 26 kpsi, about 28 kpsi, about 30 kpsi, about 32 kpsi, about 34 kpsi, about 36 kpsi, about 38 kpsi, about 40 kpsi, about 60 kpsi, about 80 kpsi, about 100 kpsi, about 120 kpsi, about 140 kpsi, about 160 kpsi, about 180 kpsi, about 200 kpsi, about 220 kpsi, about 220 kpsi, about 240 kpsi, about 260 kpsi, about 280 kpsi, about 300 kpsi, about 320 kpsi, about 340 kpsi, about 360 kpsi, about 380 kpsi, about 400 kpsi, or the Flexural Modulus can take the form of a range that is bracketed by any of the above two values. The word about, can mean plus or minus 5 percent, plus or minus 10 percent, plus or minus 15 percent, plus or minus 20 percent, plus or minus 50 percent, and so on.

In exclusionary embodiments, system, medical device, and catheter of the present disclosure can exclude any system, medical device, or catheter, where a catheter or segment thereof, a tubing or segment thereof, a sheath or a segment thereof, is definable by one of the above Flexural Modulus parameters.

Measuring hardness of entire catheter, or of a specific segment of catheter, or of transition region between two adjacent regions of catheter, or of sheath. Hardness of a plastic can be defined in terms of a “durometer” value. Hardness is defined and tested as a material's resistance to indentation. The hardness can be, for example, about 45, about 50, about 55, about 60, about 65, about 70, about 75, about 80, about 85, about 90, about 95, or about 100. In attributing any of these durometer values to a plastic substance or other substance, one must also state which scale is used. For example, the scale can be ASTM D2240 type A scale, which is used for softer materials, or the ASTM D2240 type D scale, which is used for harder materials (see, Silicon Design Manual, 6th ed., Albright Technologies, Inc., Leominster, Mass.).

The hardness of the devices of the present disclosure, including hardness of specific features, such as a tip, wall, bump, tapered region, hub, wing, tab, conical region, bead-like region, can be measured by the durometer method and Shore hardness scale. See, e.g., U.S. Pat. No. 5,489,269 issued to Aldrich, U.S. Pat. No. 7,655,021 issued to Brasington and Eleni (2011) Effects of outdoor weathering on facial prosthetic elastomers. Odontology. 99:68-76, which are each individually incorporated herein by reference in their entirety. Shore A hardness refers to hardness determined where a steel rod dents in the material, while Shore D hardness refers to hardness that is determined where a steel rod penetrates into the material. Shore hardness, using either the Shore A or Shore D scale, is used for rubbers/elastomers and is also commonly used for softer plastics such as polyolefins, fluoropolymers, and vinyls. The Shore A scale is used for softer rubbers while the Shore D scale is used for harder rubbers. Hardness by either scale can be measured with instruments from Kraiburg TPE, Buford, Ga., where durometer values can be either DIN53505 standard, or by ISO7619-1 standard.

Medical Instruments or Medical Tools that can be Passed Through the Catheter Versus Medical Instruments that are Mounted on an End of the Catheter. Balloons, fabrics for balloons, layers, adhesives, housings for balloons, devices for inserting and withdrawing balloons, related devices such as stents and catheters, methods of manufacture, and methods for administration, treatment, or diagnosis, and methods for insertion or withdrawal of a medical device from a patient, are available. See, for example, U.S. Pat. No. 7,862,575 of Tal; US 2007/0060882 of Tal, US 2011/0160661 of Elton; US 2010/03180 of Pepper). Each of these patents and published patent applications is hereby incorporated by reference as if set forth herein in its entirety.

In an exclusionary embodiment, system, medical device, catheter, of the present invention can exclude any instruments that are mounted on, or attached to, or coupled to, at the end of the catheter. This system is allowing access to the cerebrocervical vasculature so that tools can be inserted into the catheter or sheath lumen to then entire the brain vasculature to perform the intervention. In exclusionary embodiments, system, medical device, catheter, and methods of the present disclosure can exclude any system, medical device, or method, where a medical instrument or tool is mounted on distal end of the catheter, or where a medical instrument or tool is mounted on proximal end of catheter, or where a medical instrument or tool resides within the lumen of a catheter.

Methods for inserting a catheter or sheath into a blood vessel include the use of the Seldinger technique. The Seldinger technique includes the initial step of inserting a needle into a patient's blood vessel. A guide wire is inserted through the needle and into the vessel. The needle is removed, and a dilator and sheath combination are then inserted over the guide wire. The dilator and sheath combination is then inserted a short distance through the tissue into the vessel. The combination of the needle, dilator, and sheath, can be advanced over the guide wire into the blood vessel. After this combination has been advanced, the dilator is removed. The catheter is then inserted through the sheath into the vessel to a desired location. The Seldinger technique, and variations thereof, and devices used to perform this technique, are described in Seldinger (1953) Acta Radiologica 39:368-376; U.S. Pat. No. 7,722,567 issued to Tal, U.S. Pat. No. 7,972,307 issued to Kraus, et al, and U.S. Pat. No. 7,938,806 issued to Fisher, et al, which are incorporated by reference. U.S. Pat. No. 6,004,301 issued to Carter, incorporated by reference in its entirety, provides several elementary diagrams that disclose the insertion of a needle through the patient's flesh, with insertion into a blood vessel.

Exclusionary embodiments relating to Seldinger technique and electronic components. In embodiments, system, devices, medical devices, catheters, and methods can exclude any system, medical device, or method that uses Seldinger technique, or that comprises a sheath, or that comprises a guide wire.

Alternatively, embodiments, system, devices, medical devices, catheters, and methods can exclude any system, medical device, or method that uses Seldinger technique, or that comprises a sheath, or that comprises a guide wire, for purposes of the invention as defined by the present claim set, but where one or more of Seldinger technique, sheath, and guide wire can optionally be utilized by an operator, where the operator is using system, medical devices, catheters, and methods of the present disclosure.

In exclusionary embodiments, system, devices, catheters, and methods of the present disclosure can exclude electronic components, such as a battery, capacitor, light emitting diode (LED), heating element, motor, radio transmitter, electric wire, coaxial cable, electromagnet, and so on, for the purposes of the invention as defined by the present claim set, but one or more electronic components can optionally be utilized by an operator, where the operator is using system, medical devices, catheters, and methods of the present disclosure.

Copolymer Embodiments; Porosity Embodiments; Hydrogel Embodiments. Copolymers are encompassed by the disclosure, for example, copolymers of the block type and copolymers of the rake type (see, e.g., U.S. Pat. No. 8,008,407 of Oberhellman et al. and U.S. Pat. No. 8,084,535 of Maton et al, which are incorporated herein by reference in their entirety). Regarding porosity, if the porosity of a polymer coating is not sufficient to allow diffusion of an agent, such as a drug, into the extracellular fluids, a porosigen, such as lactose, can be added to the polymer used for the coating. Hydrogels, and methods for controlling water content of hydrogels, and mechanical strengths of various types of hydrogels are described (see, e.g., U.S. Pat. No. 4,734,097 of Tanabe et al, which is hereby incorporated by reference in its entirety). Because of their weak, rubbery mechanical properties, polysiloxane is sometimes prepared as chemically crosslinked, or synthesized as a block polymer that alternates with a harder type of polymer (see, page 36 of F. Wang (1998) Polydimethylsiloxane Modification of Segmented Thermoplastic Polyurethanes and Polyureas, Thesis, Virginia Polytechnic Institute and State Univ., Blacksburg, Va.)

Silicone can reduce the coefficient of friction. Methods for applying silicone and for measuring coefficient of friction are available (see, e.g., U.S. Pat. No. 5,013,717 of Solomon). What is provided is medical device that retain their “plastic memory,” such as medical device comprising thermoplastic polyurethane, as compared to vinyl resin (see, e.g., U.S. Pat. No. 4,579,879 of Flynn). What is provided is medical device that, in its entirety, or in segments, comprises siloxane. Medical device comprising siloxane has increased flexibility, when compared, for example, to a medical device that is substantially made of polyurethane (see, e.g., U.S. Pat. No. 8,092,522 of Paul et al). “Lubricity” can be quantitated in terms of the unit, coefficient of friction. “Lubricity” measures the frictional properties or tackiness of a material. A low coefficient of friction may be desired for medical devices to minimize trauma to the patient's body (see, page 226 of Vascular Medicine and Endovascular Interventions (ed. by T. W. Rooke) Blackwell Futura (2007)).

Lubricity can be provided by the polymer that is used to manufacture medical device, catheter, or sheath, of the present invention. Silicone can reduce the coefficient of friction. Methods for applying silicone and for measuring coefficient of friction are available (see, e.g., U.S. Pat. No. 5,013,717 of Solomon, which is incorporated by reference in its entirety). What is provided is medical device that retain their “plastic memory,” such as medical device comprising thermoplastic polyurethane, as compared to vinyl resin (see, e.g., U.S. Pat. No. 4,579,879 of Flynn, which is incorporated herein by reference in its entirety). What is provided is medical device that, in its entirety, or in segments, comprises siloxane. Medical device comprising siloxane has increased flexibility, when compared, for example, to a medical device that is substantially made of polyurethane (see, e.g., U.S. Pat. No. 8,092,522 of Paul et al, which is incorporated by reference in its entirety).

Reagents and Equipment Pharmaceutical agents, drugs, and other chemical reagents, and laboratory equipment, are available (see, e.g., Sigma Aldrich, St. Louis, Mo., Thermo Fisher Scientific. Waltham, Mass.). Surgical supplies, tools, and equipment, are available (see, e.g., Stryker, Kalamazoo, Mich., DiaMedical, West Bloomfield, Mich., Medtronic, Fridley, Minn.). Components for the methods and devices of the disclosure are available, for example, from Advanced Cardiovascular Systems in Santa Clara, Calif.; Baxter International of Deerfield, Ill.; Abbott Laboratories at Abbott Park, Ill., Edwards Lifesciences, Irvine, Calif., and Boston Scientific of Natick, Mass. Components of the present disclosure can be made, without limitation, by molding, blow molding, slush molding, injection molding, rotational molding, compression molding, extrusion, thermoforming, stamping, calendaring, and so on (Brazel, C S; Rosen, S L (2012) Fundamental Principles of Polymeric Materials. Wiley, Hoboken, N.J.).

A composition that is “labeled” is detectable, either directly or indirectly, by spectroscopic, photochemical, biochemical, immunochemical, isotopic, or chemical methods. For example, useful labels include 32P, 33P, 35S, 14C, 3H, 125I, stable isotopes, epitope tags fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

In exclusionary embodiments, the present disclosure can exclude any system, device, instrument, reservoir, coating, or method that contains one or more of the above agents.

In embodiments, systems, medical devices, catheters, and methods of the present disclosure, can be used for diagnosis or for treatment of aberrant vessels that form vascular rings where these vascular rings compress the trachia or compress the esophagus.

In embodiments, systems and methods of the present disclosure includes three dimensional magnetic resonance angiography, before, during, or after treating a patient (see, Krinsky, Rofsky, Weinreb (1996) Gadolinium-Enhanced Three-Dimensional MR Angiography of Acquired Arch Vessel Disease. MR. 167:981-987). In embodiments, system and methods of the present disclosure includes computed tomography, before, during, or after treating a patient (see, Boufi, Loundou, Alimi (2017) Eur. J. Vase. Endovasc. Surg. 53:663-670). In embodiments, system and methods of the present disclosure includes magnetic resonance Imaging (MRI), before, during, or after treating a patient (see, Sechtem, Fisher, Higgins (1987) AJR Am. J. Roentgnol. 149:9-13).

Guidance for placing a medical device during use in a patient, can be provided, by ultrasound or optical coherence tomography (Muraoka et al (2012) 28:1635-1641; Kang et al (2011) Circ. Cardiovasc. Interv. 4:139-145; Alfonso et al (2012) 103:441-464). Medical device can be configured, for placing at or near neointimal formation, location at risk for restenosis, atherosclerotic plaque, bile tract, urinary tract, lymphatic duct, intestines, pulmonary tract, and so on. Identifying lesions at risk for restenosis can be made by available methods (see, e.g., Montalescot et al. (1995) Circulation. 92:31-38; Killip et al (1995) J. Nuclear Med. 36:1553-1560; Garg et al. (2008) J. Am. College Cardiol. 51:1844-1853).

In exclusionary embodiments, systems, catheters, medical devices, and methods of the present disclosure can exclude any system, medical device, catheter, or method that uses three dimensional magnetic resonance angiography, that uses magnetic resonance imaging (MRI), or that uses computed tomography.

Therapies for CSH 111 and arteriovenous malformations 107 and nose bleeds/epistaxis 109 and are better secured by this access it is respectfully proposed.

Data streams, mediated by AI, with automatic default settings, related to analytics of devices, patients and system outcome.

An app compiling data accessible to physicians regarding sensual and collected trans-maxillary data.

### EXAMPLE LAST—USE WITH CONTRACTOMERES

Drawbacks of the inventor's related art from PCT/US2017/06584 were that the involved “contractomeres” or crawling catheters, were not able to be mapped out on treatment and therapy protocols. It is respectfully proposed that in the context of the present invention, those systems and processes may be used in a selected vessels now being accessed.

According to embodiments shown and described and illustrated herein there is disclosed a process, wherein during periods of “rest” or non-aspiration conditions, the proleg is flush with the main catheter and appears as a longitudinally vertebrated portion of the main catheter. During collapse of the contractomere, under axial load, the proleg components bend outward, fattening the diameter of the catheter.

According to embodiments there is disclosed a catheter system wherein he frequency of the aspiration wave can vary to augment or retard the inching of the catheter. There can be plateaus of negative pressure or positive pressure at the discretion of the operator. A low and high frequency variance changes the activity of the catheter. Low frequencies are more likely to cause forward progress, high frequencies are more likely to cause break-up of occlusive materials at the end of the catheter.

The catheter will be used as a platform for other devices deployed or in preparation for deployment in the artery. In the setting of occlusion: stents, retrievers, stent-retrieving combinations, balloons and separators can all be used in concert with the invention. These devices, commonly used in stroke and pulmonary embolism, will act as distal anchors for the “crawling” catheter. During catheter motion, it will invest and ingest the occlusive material, and ride over the anchoring devices to envelope them as well.

The advantages of this catheter are related to its use in the vascular system. Where the peristalsis of the catheter generates an atraumatic anterograde advancement. Inside the muscular artery this retains a physiologic profile in the setting of aspiration or retrieval of occlusive materials. Traditional catheters act as fixed tubular skeletons and can create force vectors that damage vessel lumens. The invention is modular and takes into account the elastic property of the artery wall.

Catheter: is commonly used to identify a tubular instrument that is inserted into a human body cavity or orifice, naturally or surgically opened.

Catheter substrate: unless otherwise indicated, a “catheter substrate” is a catheter or one or more components thereof, such as a catheter body, juncture hub, extension line or connector.

Coating: unless otherwise indicated, “coating” refers to any temporary, semi-permanent or permanent layer, or layers, treating or covering a surface. The coating may be a chemical modification of the underlying substrate or may involve the addition of new materials to the surface of the substrate. It includes any increase in thickness to the substrate or change in surface chemical composition of the substrate.

Contractomere is defined for the purposes of this invention as a modular functional unit capable of facilitating relative movement from a first to a second position as shown, for example by the two sets of arrows in the first figure—exemplary embodiments shown are not meant to be limiting and those skilled in the art understand that, for example, prolegs 232 and their functional equivalents such as stent-like structures may be substituted based on functional homologous, inter alia.

Degradation Products: unless otherwise indicated, “degradation products” are atoms, radicals, cations, anions, or molecules other than water formed as a result of hydrolytic, oxidative, enzymatic, or other chemical processes.

The term “distal” refers to a direction relatively furthest from a clinician using a catheter described herein. For example, the end of a catheter placed within the catheter body of a patient is considered a distal end of the catheter, while the catheter body of a patient is considered a distal end of the catheter, while the catheter end remaining outside the catheter body is a proximal end of the catheter.

Hydrophilic: unless otherwise indicated, “hydrophilic” refers to solvents, molecules, compounds, polymers, mixtures, materials, or functional groups which have an affinity for water. Such materials typically include one or more hydrophilic functional groups, such as hydroxyl, zwitterionic, carboxy, amno, amide, phosphate, sulfonyl, hydrogen bond forming, and/or ether groups.

Hydrophobic: unless otherwise indicated, “hydrophobic” refers to solvents, molecules, compounds, polymers, mixtures, materials, or functional groups that are repelled by water. Such materials typically contain non-polar functional groups.

The term “proximal” refers to a direction relatively closer to a clinician using a catheter described herein. For example, the end of a catheter placed within the body of a patient is considered a distal end of the catheter, while the catheter end remaining outside the body is a proximal end of the catheter.

Tip Region: unless otherwise indicated, “Tip Region,” as used herein, shall mean the terminal 10 cm length of the catheter body at the distal end of the catheter body.

Undercoating Layer: unless otherwise indicated, “undercoating layer” refers to any coating, or combination of coatings, incorporated into a substrate from which a hydrophilic polymer is grafted.

The present invention applies variable segments which are contractile to any known catheter system, in combination with fixed segments, whereby collapsible nodes enable movement of catheter 99.

The catheter body may be fabricated from any of a range of biocompatible polymers. For example, in certain embodiments the catheter body may be comprised of thermoplastic polyurethanes (“TPU”), thermoplastic polyurethane-silicones, silicones, or a combination thereof. Exemplary polyurethanes include Lubrizol Tecothane®, Lubrizol Carbothane®, Lubrizol Tecoflex®, Lubrizol Pellethane®, Lubrizol Estane®, Bayer Desmopan®, Bayer Texin®, DSM Bionate®, DSM Biospan®, DSM Bionate® II, DSM Elasthane®, BASF Elastollan™, Biomerics Quadrathane™, Biomerics Quandraflex™, Biomerics Quadrahilic™, or a blend thereof, in a range of hardnesses from 100 A to 80 A durometer. Alternatively, exemplary polyurethanes will have a range of hardnesses from 70 A to 72 D. Exemplary polyurethane-silicones include AorTech Elast-Eon™, AorTech ECSilr™, DSM CarboSil®, DSM Pursil®, or a blend thereof in a range of hardnesses from 80 A to 60 D durometer. Alternatively, exemplary polyurethane-silicones will have a range of hardnesses from 70 A to 72 D. Exemplary silicones include peroxide-cured and platinum cured silicones in a range of hardnesses from 50 A to 60 D durometer. Alternatively, exemplary silicones will have a range of hardnesses from 50 A to 70 D. Additionally, the biocompatible polymer may optionally contain a radiopacifier such as barium sulfate, bismuth trioxide, bismuth subcarbonate, bismuth oxychloride, tungsten, or tantalum, or a combination thereof. If included, the radiopacifier will typically be added at 5 wt % to 40 wt %. Colorants may also be included in the biocompatible polymer and the catheter body would then be opaque.

Referring now to the figures showing an aspiration catheter example, wherein FIG. 1 is a perspective view of a catheter 99 in accordance with one embodiment. The catheter shown is a fixed segment molded to the vessel and there are variable contractile segments or contractomeres, 103 shown is a first position, which moves to a second position 105 advancing the catheter like the movement of a common earthworm.

Each node moves from a first to a second position and allows the catheter to move axially. This version works well for high viscosity slurry types of materials.

Wave proportion and crawling are achieved as would be known by those skilled in the art.

The contractomere's jacket and spring are made separately and later bonded to the catheter. The proleg is made by barrel staving the catheter along its axis. Each contractomere is calibrated to displace at a specific force and thus a unique kinomatic profile, though there is overlap of activation of each contractomere during an aspiration cycle.

In the setting of an occluded artery, the catheter will sit in close proximity to the obstruction. With this arrangement the artery and catheter together act as a fixed hydrostatic system, with a relatively unchanged fluid volume. With cyclical aspiration, the catheter will inch toward the occlusion.

The aspiration mechanism is a device that is attached directly or indirectly to the end of the catheter. It will provide sinusoidal vacuum forces that will cause repetitive loading and unloading of forces on the contractomere. The frequency of the aspiration wave can vary to augment or retard the inching of the catheter. There can be plateaus of negative pressure or positive pressure at the discretion of the operator. A low and high frequency variance changes the activity of the catheter. Low frequencies are more likely to cause forward progress, high frequencies are more likely to cause break-up of occlusive materials at the end of the catheter.

The catheter will be used as a platform for other devices deployed or in preparation for deployment in the artery. In the setting of occlusion: stents, retrievers, stent-retrieving combinations, balloons and separators can all be used in concert with the invention. These devices, commonly used in stroke and pulmonary embolism, will act as distal anchors for the “crawling” catheter. During catheter motion, it will invest and ingest the occlusive material, and ride over the anchoring devices to envelope them as well.

The advantages of this catheter are related to its use in the vascular system. Where the peristalsis of the catheter generates an atraumatic anterograde advancement. Inside the muscular artery this retains a physiologic profile in the setting of aspiration or retrieval of occlusive materials. Traditional catheters act as fixed tubular skeletons and can create force vectors that damage vessel lumens. The invention is modular and takes into account the elastic property of the artery wall.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention.

Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A process for delivery of therapies by trans-maxillary access, which comprises the steps of:
catheterizing the superficial temporal artery (STA);
accessing at least another vessel through a retrograde approach; and
delivering therapies using wire and catheter borne treatments.

2. The process of claim 1, whereby the catheterizing step includes pre-surgical application of a cream further comprising at least a calcium channel blocker.

3. The process of claim 2, optionally comprising a forked device for stabilizing the principal or collateral vessel to facilitate device placement proximate to or within a subject lesion.

4. The process of claim 3, the principal vessel being, for example, the superficial temporal artery, frontal branch, and the forked device pinning the superficial temporal artery parietal branch in place with addressing a subdural hematoma or layer of chronic blood sitting on brain surface.

5. The process of claim 2, wherein the delivering step further comprises docking an infusion port on the scalp for long term medication infusions via needle access, and catheter linkages to the superficial temporal artery (STA) and related vessels and organs.

6. The process of claim 2, the accessing step includes advancing a catheter system into at least one target selected from the group consisting essentially of the internal carotid artery (ICA), the heart and coronary vessels, the middle meningeal artery (MMA), and the maxillary system to nose, face and eyes.

7. The process of claim 6, the catheter system being advanced to navigate via the retrograde approach into the external carotid artery (ECA) from the superficial temporal artery (STA); and, said catheter system further comprises at least a balloon catheter.

8. The process of claim 7, whereby medication may be eluted into the carotid system, namely via the external carotid artery (ECA), which would then travel into the internal carotid artery (ICA).

9. The process of claim 5, further comprising, in combination:
infusion of thrombolytic and non-thrombolytic drugs.

10. The process of claim 9, comprising:
Lidocaine for the treatment of headache; tPA/Heparin for extended stroke treatment;
calcium channel blockers, to address vasospasm, and migraine-addressing agents, namely, Rimegepant.

11. The process of claim 8, further comprising, in combination:
infusion of thrombolytic and non-thrombolytic drugs, selected from the group of Lidocaine for treatment of headache;
tPA/Heparin for extended stroke treatment;
calcium channel blockers for vasospasm, and Rimegepant (Nurtec® ODT) for migraines.

12. A system for trans-maxillary access and therapy, which comprises, in combination:
access devices for direct entry into the head and neck;
catheters specifically adapted to proximate circulation and effective to be directed to extra and intracranial pathology;
wires effective for retrograde size and tortuosity of intracranial networks;
direct access and closure devices supporting the novel portal of entry; and,
sensing technology along with specialized computing apparatus and connection/transmission hardware to harvest, array, and select data generated by and used to drive wires and catheters unique to this space.

13. The system of claim 12, said specifically adapted catheters, ranging between at least about 28 and 42 cm in length, and having greater Young's Modulus type of degrees of flexibility than existing products on the market, and including smaller balloon tip catheters.

14. The system of claim 12, said direct access and closure devices further comprising:
shortened wires, ranging from at least about 48 to 77 cm in length, and ultra-short sheaths and introducers.

15. The system of claim 13, further comprising an implantable, long-term infusion port, having needle access, at least a pump and reservoir operatively linked to catheters linked to the superficial temporal artery (STA), where the port is docked on the scalp, and provides for chronic access to therapies in pharmaceutical form.

16. The process of claim 2, for treating chronic subdural hematomas by accessing the middle meningeal artery (MMA) for more direct entry into the head by locating/imaging the superficial temporal artery (STA) just under surface of the scalp as a retrograde access site.

17. The process of claim 6, effectively used to treat epistaxis (nose bleeds).

18. The process of claim 6, wherein the retrograde approach treats arteriovenous malformations within the external carotid artery (ECA), the internal carotid artery (ICA), the middle meningeal artery (MMA), and/or the maxillary system.

19. The process of claim 6, wherein the heart and coronary vessel are addressed.

20. The process of claim 19, the wires/microwires navigating in a retrograde fashion enable treatment by travelling up the middle meningeal artery (MMA) and down the superficial temporal artery (STA).

21. A process for retrograde access of the distal portion of the neurovasculature, which comprises, in combination:
imaging or otherwise locating the occipital artery;
catheterizing the same following application of locally administered calcium channel blocker;
addressing vasculature issues via retrograde microwire/catheter/balloon/stent-like device combinations; and
using specialized catheter sensing devices, optionally on chips, and closure devices and repeating as needed.

22. Products by the process of claim 21, further comprising data sets of individuated vessel groupings, patient groupings and gross morphology sets including registration of images and pressures.

23. The system of claim 20, comprising at least a sensor and microelectronics support communicating in a database.

24. The system of claim 20, further comprising:
a contractomere-based process for advancing and stopping catheters, which comprises, in combination:
employing plateaus of negative pressure and positive pressure;
using low and high frequency variance changes;
causing balancing against movement with said delta frequency.

25. Catheter mobilizing contractomeres according to claim 9, comprising; in combination:
at least two nodes incorporated into a catheter or similar material device further comprising:
a coiled spacing emplaced against a wall as single or double or multiple helical configuration;
a pleated jacket, matching the helical or screen itch of the spacings; and
a proleg extension of the non-contractomeres, or fixed component of the catheter involved the jacket and spacing.

26. Catheter mobilizing contractomeres of claim 10, wherein the coiled spacing comprises stainless steel and stores energy during negative vacuum.

27. Catheter mobilizing contractomeres of claim 11, further comprising biocompatible polymers forced with Nitinol® braids to provide strain relief and reduce kinkability.

28. The system of claim 14, further comprising contractomeres.
